(19) United States
(12) Patent Application Publication
(10) Pub. No.: US 2021/0099849 A1
(43) Pub. Date: Apr. 1, 2021

(54) EMERGENCY SYSTEM FOR A VEHICLE

(71) Applicant: Harman Becker Automotive Systems GmbH, Karlsbad (DE)

(72) Inventors: Richard Zoidl, Schweitenkirchen (DE); Anilkumar Hariharakrishnan, Sauerlach (DE); Stephan Preuss, Schwabhausen (DE)

(73) Assignee: Herman Becker Automotive Systems GmbH, Karlsbad (DE)

(21) Appl. No.: 17/029,749

(22) Filed: Sep. 23, 2020

(30) Foreign Application Priority Data
Sep. 27, 2019 (DE) 102019126208.9

(51) Int. Cl. H04W 4/44; H04L 9/32; G16H 50/30; G16H 80/00; B60W 60/00

(52) U.S. Cl. CPC H04W 4/44; H04L 9/3231; G16H 50/30; B60W 2756/10; B60W 60/0016; B60W 2540/221; B60W 2556/45; G16H 80/00

(57) ABSTRACT

An emergency system (100, 200) for a vehicle comprising a data collection unit (101) for recording emergency data, wherein the emergency data comprise at least vehicle data (110) and the medical data (120) of at least one user of the vehicle, a data storage unit (140) for storing the emergency data, and a data access unit (150) by means of which a communication link (182) can be established to the data storage unit (140).

### CROSS-REFERENCE

Priority is claimed to application Ser. No. 102019126208.9, filed Sep. 27, 2019 in Germany, the disclosure of which is incorporated in its entirety by reference.

### TECHNICAL FIELD

The invention relates to an emergency system for a vehicle.

### BACKGROUND

An automatic emergency call system, known as eCall, is mandatory for passenger cars type-approved in the EU starting April 2018. After an accident, this emergency call system automatically or even manually establishes a voice telephone link with an emergency call center. In addition, further data can be transmitted such as vehicle-relevant data, the current position, and the event that triggered the emergency call. This emergency call system enables rapid alerting of rescue vehicles.

An object of the invention is to obtain an improved emergency system.

### SUMMARY

This object is achieved according to the invention by means of an emergency system having the features listed in claim 1 and by means of a method with the features listed in claim 26.

The emergency system has a data collection unit for recording emergency data, wherein the emergency data comprise at least vehicle data and the medical data of at least one user of the vehicle. It further has a data storage unit for storing the emergency data and a data access unit, by means of which a communication link to the data storage unit can be established.

In contrast to the prior art, the emergency system comprises not only the vehicle data but also the medical data of a user of the vehicle. This offers the advantage that conclusions can be drawn about the type and severity of the injuries to the vehicle users due to the transmission of the medical data. Moreover, the number of vehicle occupants can be known early on. In this manner, not only the correct type but also the necessary number of rescue vehicles can be provided.

In addition to the medical data, there is a transmission of the vehicle position which means that the emergency personnel can be routed to the correct location. Thus, a substantially more targeted use of the emergency personnel can be implemented, whereby lives can be saved, particularly in especially critical cases.

Expediently, the communication link is formed for voice and/or data transmission. It is especially suitable in this case when a wireless communication link is available. This can take place, for example, via an existing mobile network.

In one or more embodiments, there can be means available for identifying the at least one user. It can thereby be ensured that all vehicle users which are present in the vehicle, for example, at the time of an accident are detected, and there is medical data available for all users. The identification can take place in different ways. For example, the driver could be identified by means of the personalized vehicle key used.

Identification can also take place via a portable device. For example, the mobile telephones of the users could be connected to the emergency system so that all vehicle users are known.

It is especially advantageous, however, when there are means available for the automatic identification based on unique personal characteristics, for example pulse, pupil, or fingerprint. This can take place, for example, via one or more cameras in the vehicle interior. In this manner, it can be ensured that all vehicle users are known at all times in order to have the relevant data available in an emergency.

Due to the sensitive nature of the data present with medical data, it is expedient when the access to this is controlled and/or limited in a special manner. The emergency system can thus have a foreground mode and a background mode, wherein the communication link can be established in the foreground mode and wherein the emergency system is in the background mode during normal operation. In this manner, general access to the emergency data and particularly the medical data is prevented.

In one or more embodiments, the emergency system has at least one trigger mechanism, with which the emergency system can be switched to the foreground mode.

It may be advantageous when the emergency system has a multistage, particularly three-stage, trigger mechanism. In particular, a manual trigger mechanism and a completely automatic trigger mechanism may be present. A user can switch to the foreground mode via the manual trigger mechanism when the user detects an emergency. The automatic trigger mechanism can be activated, for example, by vehicle sensors in the event of an accident. Furthermore, a semi-automatic trigger mechanism, for example, may be present which requires a manual confirmation.

In one or more embodiments, the emergency system has a manual trigger mechanism which can be controlled by a user. In this manner, an emergency situation can be reported independently of an accident or a medical emergency.

In one or more embodiments, the emergency system has a vehicle trigger mechanism, which can be controlled by the vehicle, wherein a confirmation by a user is necessary. The vehicle trigger mechanism can activate automatically, for example, when a less serious emergency has been detected. However, a user must confirm the emergency before there is a switch into the foreground mode. In this case, the trigger mechanism can be configured such that there is an automatic trigger or even no trigger after a certain confirmation time has expired without such a confirmation. This may also depend on the type of emergency determined.

In one or more embodiments, the emergency system has an emergency trigger mechanism, which can be controlled automatically and autonomously by the vehicle, particularly when an emergency is determined. The trigger mechanism in this case is triggered automatically based on the determination of a significant emergency, such as an accident. An intervention by a user is not possible in this case.

In one or more embodiments, the trigger mechanism may be formed by the data collection unit. Because the data collection unit processes all of the relevant vehicle and medical data, a simple classification of the alarm trigger can occur here.

In one or more embodiments, the data collection unit is connected to an interface for vehicle data for this purpose. For example, vehicle sensors or other vehicle-relevant data can thereby be collected.

In this case, essentially all relevant data of the vehicle can be read and collected. Such an interface can be implemented, for example, on a vehicle bus, such as the CAN bus.

The collected vehicle data may comprise, for example, the following: Accident sensor data and accident data logs, airbag status data, seat position, images of the interior, particularly 2D images, 3D images, and/or infrared images.

In particular, the evaluation of an accident sensor or the statuses of the airbags can be applied for an automatic activation of the trigger mechanism.

In one or more embodiments, the data collection unit is connected to an interface for medical data. This enables access to medical data which can be transmitted to the data collection unit via the interface.

The interface for medical data may be connected, for example, to vehicle sensors, particularly for recording real-time medical data and/or dynamic medical data of at least one user. Such vehicle sensors may be, for example, fatigue sensors, alcohol sensors, interior cameras, or other sensors. Relevant medical data on the state of a user or several users can thereby be collected.

Preferably, the interface for medical data can be connected to at least one remote source for medical data. A remote source can be any source for medical data which is not permanently installed in the vehicle or a source which is connected to the vehicle. A remote source for medical data can be, for example, a home health system, a mobile and/or portable and/or implanted medical monitoring device and/or a smart phone. In particular, the remote source can be assigned to a user of the vehicle.

A remote source may also be, however, a database which is connected to the Internet and in which the medical data from portable or stationary fitness devices are stored. The medical data from remote sources may comprise dynamic, quasistatic, and static medical data. A remote source may also be, for example, an electronic medical record of a user.

The medical data may comprise, inter alia, values related to age, sex, height, weight, blood type, body mass index (BMI), allergies, illnesses, implants, pulse, blood pressure, blood glucose, blood transfusion history, transplant history, seeing and/or hearing aids, pacemakers, blood alcohol, and/or the influence of drugs.

In particular, it is advantageous when the emergency data comprise the medical data of all identified users. In this manner, targeted and rapid assistance can be provided for all vehicle users in an emergency, for example in an accident.

In one or more embodiments, the data collection unit is connected to a position unit for detecting the vehicle position, wherein the vehicle position is part of the emergency data. In this manner, the vehicle position is available for targeted alerting of emergency personnel.

Expediently, the position unit for detecting the vehicle position is connected to a satellite navigation unit and/or to a camera for monitoring the environment. Particularly autonomous-driving vehicles have wide-ranging environmental sensors, such as LIDAR or laser scanners, which can be used for more precise locating of the vehicle. In addition, further information regarding the accident can be recorded in this manner, such as persons involved in the accident outside of the vehicle.

In one or more embodiments, an emergency call center is available which is connected to the data access unit. In foreground mode, accident data can be transmitted to the emergency call center and/or can be retrieved by the emergency call center via the communication link. The emergency call center obtains access to the data storage unit and the emergency data stored therein via the data access unit.

Because the data storage unit may contain comprehensive and sensitive data regarding the users, it is advantageous when the emergency call center requires a one-time authentication for access to the data storage unit. Such an authentication can take place, for example, by means of a private-law contract. The access can be secured via an authentication such that the data storage unit cannot be accessed indiscriminately.

In one or more embodiments, at least the medical data transmitted via the communication link are encrypted. This can prevent undesired access to the emergency data. In particular, the medical data of each user can be encrypted individually, for example, with a biometric key and/or with a different key. The medical data can also be provided with a, particularly biometric, signature in order to indicate the authenticity of the data.

In one or more embodiments, the emergency system has an analysis unit, which is arranged between the data collection unit and the data storage unit and is formed for local and/or remote preparation of the data collected by the data collection unit. The analysis unit can be used to prepare the collected data. In this case, it is also possible for the preparation to take place externally or remotely. For example, substantially more computing capacity could be available in a remote computing center in order to carry out complex computations and correlations with the emergency data. The collected emergency data could also be used to train an artificial intelligence element, which enables significantly more precise accident analysis or interpretation of the collected data.

It is especially advantageous when the collected and possibly analyzed emergency data are stored in the data storage unit. The data storage unit in this case may comprise a local and/or remote data storage. In this case, it is also possible for the data storage to be connected via the Internet.

In one or more embodiments, the emergency system has a correlation unit which is formed for determining and providing correlation data. Such correlation data may comprise, for example, time, geographic position, road conditions, purpose of the trip, starting location, and/or destination. For example, it may be relevant for evaluating an emergency situation whether a user of the vehicle, for example, was just in a bar and is possibly inebriated and tired or, for example, is driving back home from a fitness studio and is thus more alert and awake. The correlation unit can establish a greater context in this case than would be possible solely from vehicle data and medical data. The correlation unit in this case can possibly also access social media and/or other platform data.

In one or more embodiments, the correlation unit is connected to the data access unit such that, in foreground mode, the correlation data can be accessed. In this manner, an emergency call center can also access the correlation data. The correlation data can also be considered, however, a part of the emergency data and be stored in the data storage unit together with the other emergency data.

In one or more embodiments, an access to the medical data of a user can also be granted by the user outside of an emergency situation. In particular, it can be advantageous in this case if the user can stipulate what access an emergency call center will have to the medical data.

In one or more embodiments, the emergency system may be part of a so-called cyber-physical system (CPS). Due to a strong network of many vehicles, accident prevention can also take place, for example, due to interventions in the route selection or the driving dynamics of a vehicle, particularly with autonomous or semi-autonomous vehicles.

A method for providing a rescue means for users of a vehicle is characterized in that an emergency call center obtains access to the emergency data of the users due to a trigger mechanism and in that the emergency call center alerts the rescue means suitable as a function of the emergency data. The provision of the emergency data means that all relevant data is present so that the emergency call center can provide a suitable number and type of rescue means. The advantage is that precious time is not lost at an accident site with detection of the situation and subsequent alerting of further rescue means.

In one or more embodiments, the emergency data comprise particularly vehicle data and medical data of the users. It is especially advantageous in this case when the medical data relate to all users so that a precise alerting is possible.

In one or more embodiments, the emergency call center can consider additional correlation data when selecting the rescue means. The correlation data can detect, for example, the context surrounding a trip and an emergency. This data can include information as to whether the trip has occurred late at night after a visit to a bar or club or during the day from or to the workplace. The evaluation of the medical data present, for example, can thereby change. For example, a higher pulse may be normal during a trip from a fitness studio or exercising while it could indicate a medical situation in a different context. The correlation data can be derived from various sources such as, for example, location data, position data, sensor data, or also from social media.

In one or more embodiments, the method is designed for using a previously described emergency system.

### BRIEF DESCRIPTIONS OF THE DRAWINGS

The system and the method are described in greater detail in the following with reference to the enclosed drawings.

The following is shown:

FIG. 1:a block diagram of a first embodiment of an emergency system;

FIG. 2:a block diagram of a second embodiment of an emergency system;

FIG. 3:a trigger diagram of an emergency system;

FIG. 4:a block diagram of various trigger mechanisms of the emergency system;

FIG. 5:a flowchart of a method for triggering an emergency;

and

FIG. 6:a flowchart of a method for alerting a rescue means after the triggering of an emergency.

### DETAILED DESCRIPTION

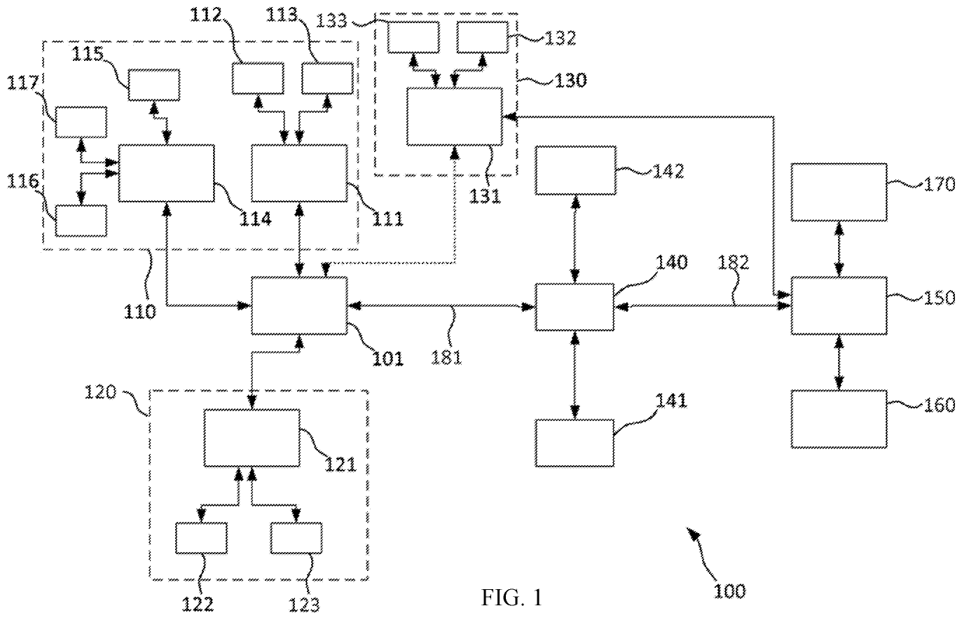

FIG. 1 shows a block diagram of an emergency system 100 according to a first embodiment. The emergency system 100 has a data collection unit 101, which is formed for collecting and combining emergency data. The data collection unit 101 may be arranged, for example, within a vehicle. However, it is also possible for the data collection unit 101 to be arranged outside of a vehicle and/or in a portable device.

The emergency data in the example comprise vehicle data 110, medical data 120, and correlation data 130.

In order to record vehicle data, the emergency system 101 has an interface 114 for vehicle data 110, which is connected to the data collection unit 101. The interface 114 is preferably arranged within a vehicle and expediently has access to data and records of an accident sensor 115, airbag and seat settings 116, as well as interior cameras 117. The interior cameras in this case can be designed for recording 2D, 3D, and/or infrared images. The type and severity of an accident can be determined solely from the information as to whether and which airbags have been deployed. The seat sensors can provide information, for example, as to how many users are in the vehicle. These data are thus very important for estimating an accident situation.

The emergency system 100 further comprises a position unit 111, which is connected to the data collection unit 101 in the example. However, it may also be advantageous when the position unit is connected to the interface for vehicle data 114, and the vehicle data collected are transferred to the collection unit. The position unit 111 is connected to a satellite navigation unit 112 and/or to environment cameras 113, which are used to determine the current vehicle position. Particularly modern passenger cars frequently have so-called 360° cameras which can also provide, for example, an impression of the accident environment. For example, it can be determined whether and possibly how many pedestrians or bicyclists, who may not have any emergency system, are involved in the accident.

In order to record medical data 120, the emergency system has an interface 121 for medical data 120, which is connected to the data collection unit 101. On the one hand, the interface 121 for medical data 120 is connected to vehicle sensors 122 for medical data 120. These vehicle sensors 122 can provide, for example, real-time data such as, for example, pulse, oxygen saturation, or respiration rate. The vehicle sensors can also provide dynamic medical data. The vehicle sensors 122 can be integrated, for example, into seats, seatbelts, armrests, headrests, or into the steering wheel and can function via direct contact or without contact. To this end, the vehicle sensors 122 may also have lasers, cameras, and/or stereo cameras.

The interface 121 can also be connected to remote sources 123 for medical data 120, for example, via a wireless communication link. Remote sources 123 may contain dynamic, quasistatic, and/or static medical data 120. A remote source may be, for example, a portable and/or implanted device, a stationary home emergency system, or even an electronic medical record of the user. The interface 121 can communicate with any data sources in many different ways in this case, such that a comprehensive medical profile of a user is available.

The medical data in this case may comprise values related to age, sex, height, weight, blood type, body mass index (BMI), allergies, illnesses, implants, pulse, blood pressure, blood glucose, blood transfusion history, transplant history, seeing and/or hearing aids, pacemakers, blood alcohol, and/or the influence of drugs. The list can be supplemented with any medical data depending on the availability of data and is not limited to the examples mentioned herein.

The data collection unit 101 is connected to a data storage unit 140, in which the collected emergency data are stored. The data storage unit 140 may have a local data storage 141 and/or a remote data storage 142. A remote data storage 142 may be, for example, a cloud storage accessible via the Internet.

In order to access the emergency data, the emergency system 100 has a data access unit 150 which is connected to the data storage unit 140 via a communication link 182. The data storage unit 140 can be permanently installed in the vehicle or may be arranged in a portable device. However, the data storage unit 140 may also be arranged stationary outside of a vehicle. The connection 181 between the data collection unit 101 and the data storage unit can accordingly be wired and/or wireless. For this reason, the communication link 182 between the data storage unit 140 and the data access unit 150 may also be wired and/or wireless. In this case, various technologies can be used, which is why the application is not limited to a specific connection.

In order to record correlation data 130, the emergency system 100 has a correlation unit 131 in the example. To this end, the correlation unit 131 may be connected, for example, to a device for recording the road conditions 132 and/or to a different source 133 for correlation data 130. Correlation data 130 may also comprise, for example, information from social networks or other Internet platforms. The correlation data enable, for example, a more precise interpretation of the collected emergency data. Thus, it can be determined, for example, from the position and the link to other data, that a user was just in a fitness studio and accordingly a pulse determined to be higher is normal and does not represent any medical indication. In this manner, different situations may also possibly be evaluated differently than the pure data situation permits.

The correlation unit 131 is connected directly to the access unit 150 in the example. This means that the correlation data is not in the data storage unit 140 but can be read out directly. In an alternative embodiment, the correlation unit 131 may also be connected, however, to the data collection unit 101 such that the correlation data 130 are part of the emergency data.

The data access unit 150 is the interface via which, for example, an emergency call center 160 and/or a trauma center 170 can obtain access to the emergency data. However, this access is only possible when the emergency system 100 is in foreground mode. The foreground mode is activated by a trigger mechanism.

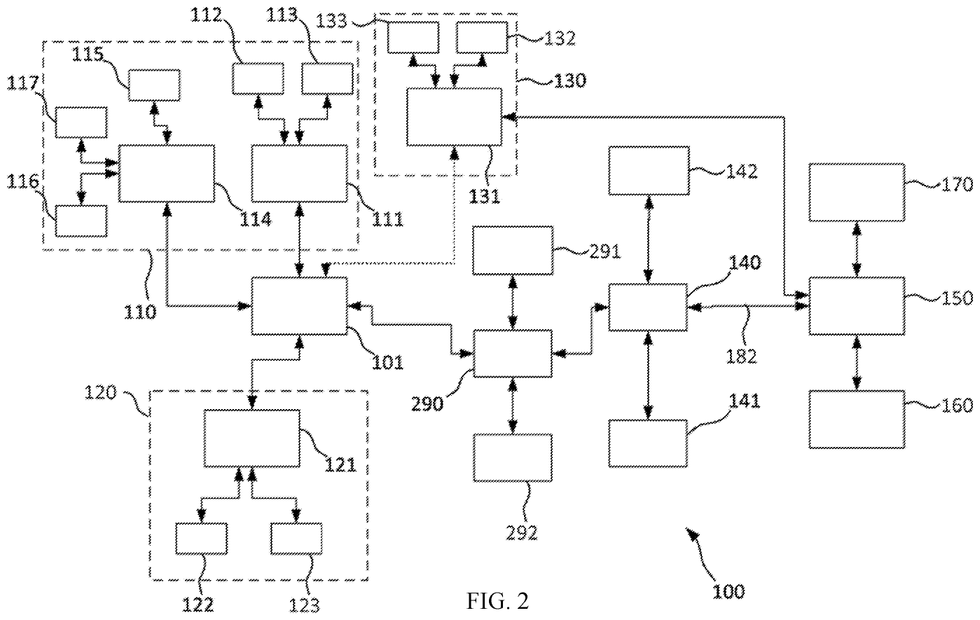

The emergency system 200 shown in FIG. 2 is essentially identical to the emergency system from FIG. 1. Thus, equivalent features are provided with the same reference numeral. The emergency system 200 additionally has, however, an analysis unit 290, which is arranged between the data collection unit 101 and the data storage unit 140. The analysis unit 290 may have a local computing unit 291 and/or a remote computing unit 292, in which the collected emergency data can be analyzed.

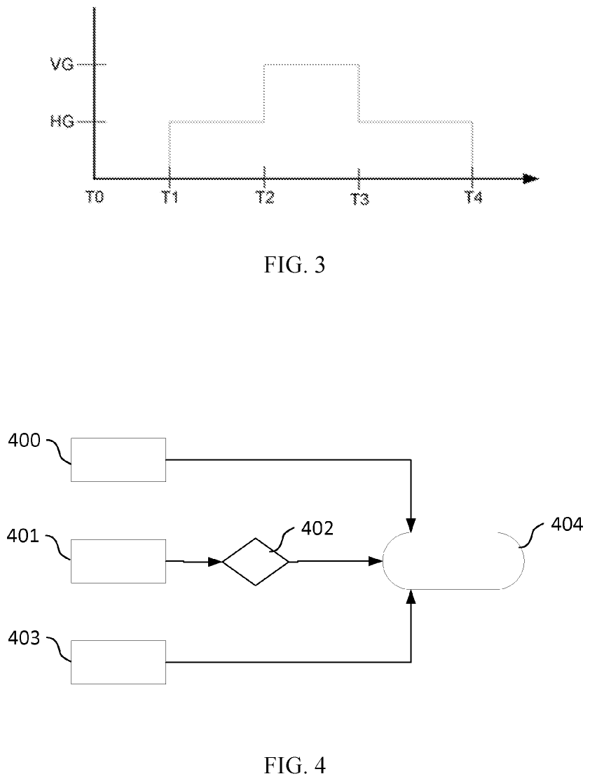

FIG. 3 schematically shows the various states of the emergency system 101. The emergency system is OFF at point in time T0. This is the case, for example, when the vehicle is switched off. The vehicle is switched on at point in time T1. The emergency system is thereby activated and switched to background mode (HG). In background mode, it is not possible to establish a communication link via the data access unit. At point in time T2, a trigger mechanism is activated, whereby the emergency system is switched to foreground mode (VG). In foreground mode, a communication link is established between the data access unit and the data storage unit, by means of which, for example, an emergency call center can retrieve relevant emergency data or the data can be transferred thereto.

FIG. 4 shows various trigger mechanisms of an emergency system. The emergency system in this case has a manual trigger mechanism 400. The manual trigger mechanism can be activated manually by a user in order to report, for example, a situation which was not detected as an emergency by the data collection unit based on the collected data.

The emergency system additionally has a semi-automatic trigger mechanism 401. In this case, the emergency system detects an emergency based on the collected data but it is not critical or requiring immediate action. With this trigger mechanism 401, a manual confirmation 402 by a user is required in order to actually activate the trigger mechanism. A maximum wait time can be specified for the confirmation. Once the wait time has expired without manual confirmation, the emergency system can be configured such that the trigger mechanism is activated or not activated. This can depend, for example, as well on the type of emergency supposedly detected.

The emergency system further has an automatic and autonomous trigger mechanism 403, which is activated by the emergency system. These can be clearly determinable emergencies such as, for example, an accident in which the vehicle sensors provide clear signals. This can be determined, for example, by an explicit accident sensor, rollover sensor, or deployed airbags.

If one of the trigger mechanisms is activated and possibly confirmed, the emergency system switches to foreground mode 404.

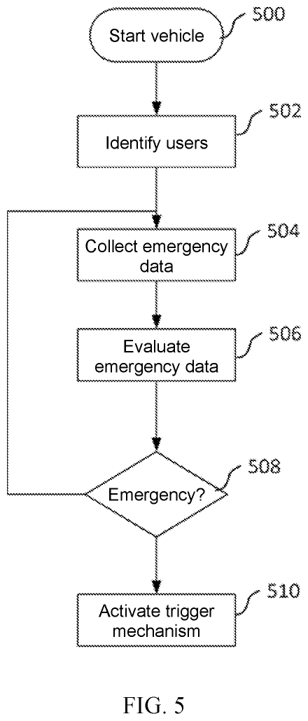

FIG. 5 shows a flowchart of a method for triggering an emergency. First, the vehicle is started in one step 500. The emergency system is thereby switched into background mode and thus activated. Then the users of the vehicle are identified 502. This can take place through biometric detection or through other features. During the trip, emergency data of all users are accordingly then continually collected 504. In this case, the emergency data can be constantly updated, for example, through a readout of vehicle sensors. The emergency data are then evaluated 506 in order to detect an emergency. After evaluation of the emergency data, there is a check 508 to determine if an emergency is at hand. If this is not the case, further emergency data are collected 504. If an emergency is detected, a trigger mechanism is activated 510, as is explained, for example, in FIG. 4.

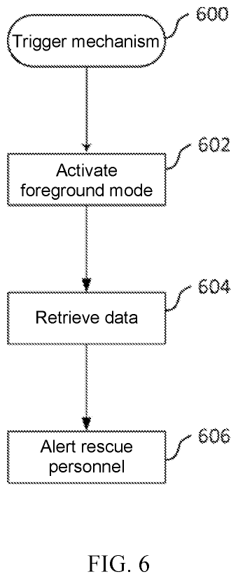

FIG. 6 shows a flowchart of a method for alerting a rescue means after the activation of a trigger mechanism 600. The emergency system activates 602 the foreground mode, whereby a communication link is established between a data access unit and the data storage unit. An emergency call center can then retrieve the emergency data 604 and alert suitable rescue means 606.

What is claimed is:

1. An emergency system for a vehicle comprising:
a data collection unit for recording emergency data, wherein the emergency data comprise at least vehicle data and medical data of at least one user of the vehicle;
a data storage unit for storing the emergency data; and
a data access unit having means for establishing a communication link to the data storage unit.

2. The emergency system according to claim 1, further comprising means for automatic identification of the at least one user based on one or more unique personal characteristics selected from the group consisting of: pulse, pupil, and fingerprint.

3. The emergency system according to claim 1 wherein, the emergency system further comprises:
a foreground mode;
a background mode;
wherein during normal operation, the communication link is established in the foreground mode and the emergency system is in the background mode during normal operation; and
wherein at least one trigger mechanism with which the emergency system can be switched to the foreground mode.

4. The emergency system according to claim 3, wherein the at least one trigger mechanism further comprises a multistage trigger mechanism having a manual trigger mechanism, a semi-automatic trigger mechanism which requires a manual confirmation, and a completely automatic trigger mechanism.

5. The emergency system according to claim 1 wherein the emergency system further comprises:
a manual trigger mechanism controlled by a user;
a vehicle trigger mechanism controlled by the vehicle and requiring a confirmation by a user; and
an emergency trigger mechanism controlled automatically and autonomously by the vehicle when an emergency is determined.

6. The emergency system according to claim 1, wherein the data collection unit is connected to an interface for vehicle data, the vehicle data is selected from the group consisting of: accident sensor data and accident data logs, airbag status data, seat position, images of the interior, the images of the interior may be 2D, 3D or infrared images.

7. The emergency system according to claim 1, wherein the data collection unit is connected to an interface for medical data.

8. The emergency system according to claim 7, wherein the interface for medical data is connected to vehicle sensors that record real-time medical data and dynamic medical data of the at least one user.

9. The emergency system according to claim 7, wherein the interface for medical data is connected to at least one remote source for medical data, wherein the medical data comprise dynamic, quasi-static, and static medical data.

10. The emergency system according to claim 9, wherein the remote source for medical data is a home health system, a mobile or portable fitness monitoring device, or a smart phone.

11. The emergency system according to claim 1, wherein the medical data is selected from the group consisting of: values related to age, sex, height, weight, blood type, body mass index (BMI), allergies, illnesses, implants, pulse, blood pressure, blood glucose, blood transfusion history, transplant history, seeing aids, hearing aids, pacemakers, blood alcohol, and the influence of drugs.

12. The emergency system according to claim 1, wherein the emergency data further comprises the medical data of all identified users.

13. The emergency system according to claim 1, wherein the data collection unit is connected to a position unit for detecting a vehicle position and the vehicle position is part of the emergency data.

14. The emergency system according to claim 13 wherein the position unit for detecting the vehicle position is connected to a satellite navigation unit or to a camera for monitoring an environment.

15. The emergency system according claim 3, further comprising an emergency call center connected to the data access unit, and in foreground mode, emergency data can be transmitted to the emergency call center or retrieved by the emergency call center via the communication link.

16. The emergency system according to claim 15, wherein the emergency call center requires a one-time authentication for access to the data storage unit.

17. The emergency system according to claim 1, wherein at least the medical data of each user transmitted via the communication link are encrypted individually with a biometric key or with a biometric signature.

18. The emergency system according to claim 1, further comprising an analysis unit arranged between the data collection unit the data storage unit, the analysis unit is formed for the local or remote preparation of the data collected by the data collection unit.

19. The emergency system according to claim 1, further comprising a correlation unit for determining and providing correlation data selected from the group consisting of: time, geographic position, road conditions, purpose of the trip, starting location, and destination.

20. The emergency system according to claim 3, further comprising:
a correlation unit for determining and providing correlation data selected from the group consisting of: time, geographic position, road conditions, purpose of the trip, starting location, and destination; and
the correlation unit is connected to the data access unit such that the correlation data that are part of the emergency data can be accessed in the foreground mode.

21. The emergency system according to claim 1, wherein access to the medical data of a user can be granted by the user outside of an emergency.

22. The emergency system according to claim 1, wherein the user can determine what medical data an emergency call center can access.
